(19) United States
(12) Patent Application Publication
(10) Pub. No.: US 2012/0150216 A1
(43) Pub. Date: Jun. 14, 2012

(54) Distally Actuated Surgical Instrument

(76) Inventors: Dyson W. Hickingbotham, (US); David F. Booth, (US)

(21) Appl. No.: 13/324,771

(22) Filed: Dec. 13, 2011

Related U.S. Application Data
(60) Provisional application No. 61/422,592, filed on Dec. 13, 2010.

(51) Int. Cl. A61B 17/28

(52) U.S. Cl. 606/206

(57) ABSTRACT

A surgical instrument for easy use of an operative tool. The surgical instrument includes a handle, a spring housing assembly, a distal assembly, a ball ring assembly, and the operative tool. The spring housing assembly is disposed at a distal end of and secured within the handle. The distal assembly is disposed against the spring housing assembly, and the ball ring assembly includes a plurality of balls. The distal assembly is positioned between the spring housing assembly and the distal assembly. The operative tool inserted through the distal assembly and connected to the spring housing assembly.

### CROSS-REFERENCE TO RELATED APPLICATIONS

This application is claims benefit of the filing date under 35 U.S.C. §119(e) of Provisional Patent Application No. 61/422,592, filed Dec. 13, 2010.

### FIELD OF THE INVENTION

The invention relates to surgical instrument and in particular to distally actuated ophthalmic surgical instrument.

### BACKGROUND

During ophthalmic surgical procedures, it is often necessary to dissect, cut delaminate or otherwise manipulate delicate tissues within the eye. Miniaturized instruments such as, for example forceps, scissors, and the like have been developed in order to allow a surgeon to operate on and within the patients eye. Known devices use plunger like components which are generally actuated by finger operated levers. More recently, handheld instruments have allowed for actuation of a distally mounted tool by application of radial force about the instruments applied around the circumference. These instruments are known as being omniactuable. Examples of such instruments are shown in U.S. Pat. No. 6,488,695 and U.S. Pat. No. 6,391,046 wherein circumferential actuators are radially displaceable in response to compression forces to operate a distally mounted tool. There is a need, however for a more simple, inexpensive actuator handle for such tools which provides smoother and effortless actuating mechanism motion.

### SUMMARY

Accordingly, the invention was devised in light of the problems described above, the invention is a distally actuated surgical instrument provide a technician or practitioner easy use of an operative tool disposed at a distal end of the instrument. Specifically, the surgical instrument includes a handle, a spring housing assembly, a distal assembly, a ball ring assembly, and the operative tool. The spring housing assembly is disposed at a distal end of and secured within the handle. The distal assembly is disposed against the spring housing assembly, and the ball ring assembly includes a plurality of balls. The distal assembly is positioned between the spring housing assembly and the distal assembly. The operative tool inserted through the distal assembly and connected to the spring housing assembly.

### BRIEF DESCRIPTION OF THE DRAWINGS

The invention will now be described by way of example with reference to the accompanying figures of which:

FIG. 1 is a perspective view of the surgical instrument according to the invention;

FIG. 2 is a cross sectional view taken along the line 2-2 of FIG. 1;

FIG. 3 is an exploded side view of the instrument of FIG. 1; and

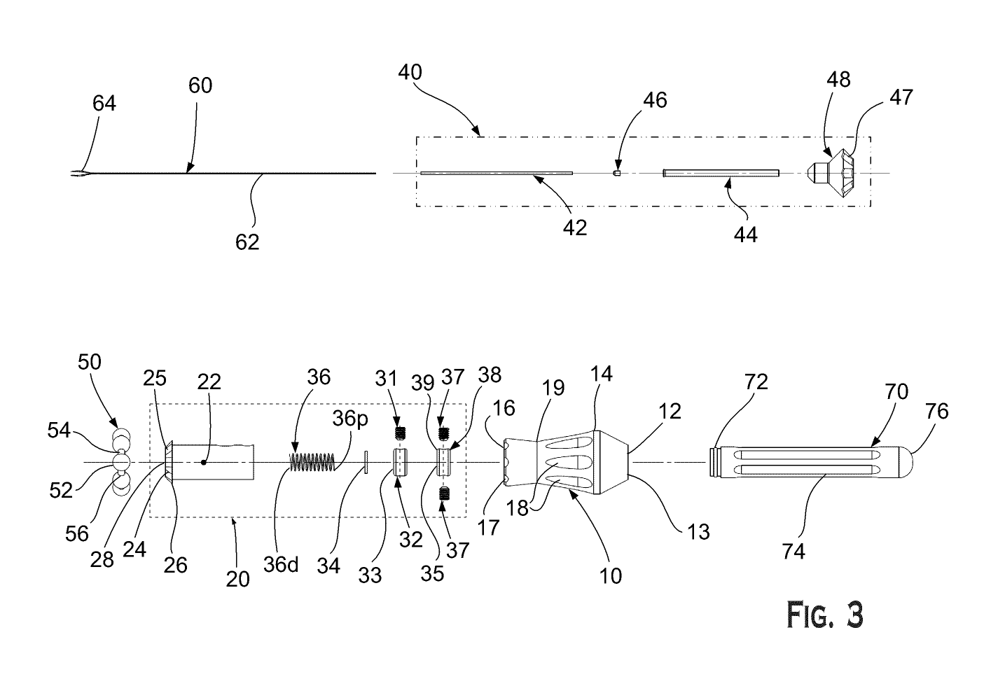

FIG. 4 is an exploded perspective view of a ball ring and rail arrangement used in the instrument of FIG. 1.

### DETAILED DESCRIPTION OF THE EMBODIMENT(S)

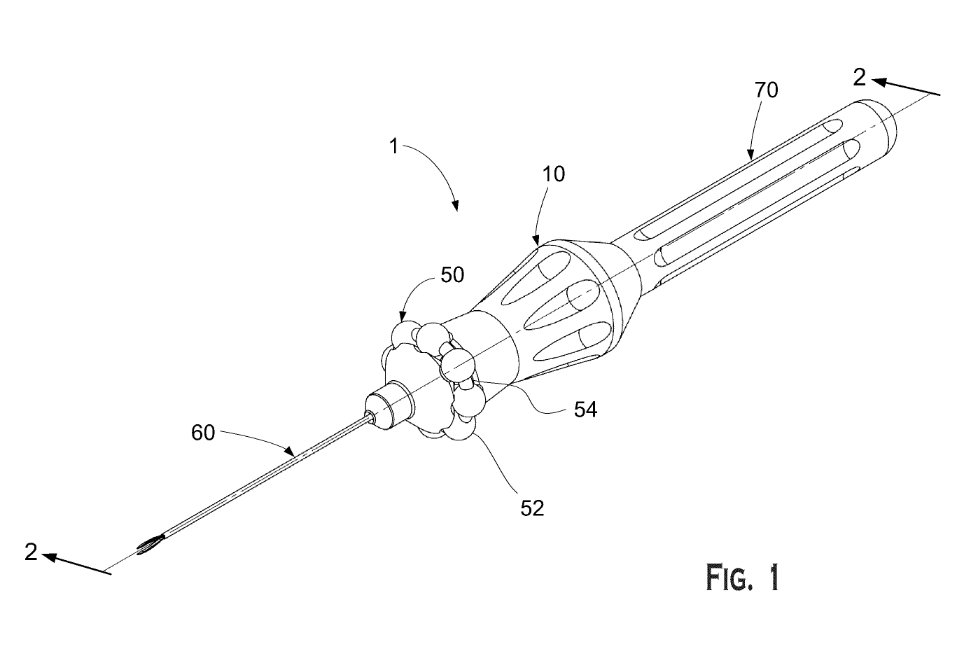

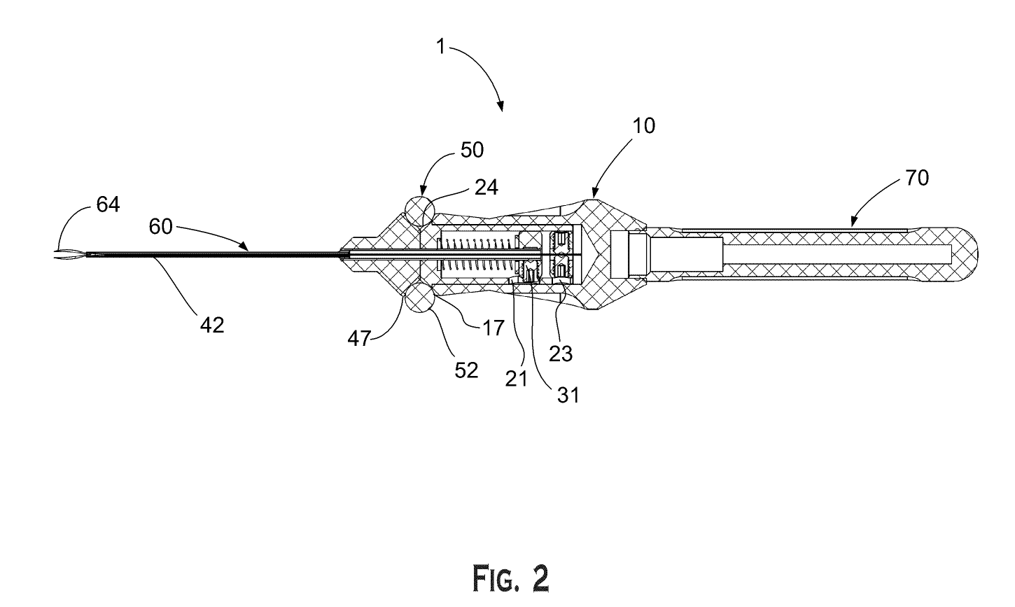

The invention will now be described in greater detail with reference to the embodiment shown in the accompanying figures. First, with reference to FIGS. 1 and 2, major components of the instrument 1 include a handle 10, a spring housing assembly 20, a distal assembly 40, a ball ring assembly 50, an operative tool 60, which in this case is shown as jaws, and an optional handle extension 70.

Now each of the major components will be described in greater detail. First, a handle 10 is a generally hollow cylindrical component having a profile which is suitable for grasping by a surgeon. In this case, its profile begins at a proximal end with a small proximal diameter 12 which tapers outward to a larger proximal area 14. A threaded opening 13 or a proximal opening with a groove that accommodates a raised ring in the distal end of an optional extension handle 70 to form a snap fit between handle 10 and optional extension handle 70 holding these parts in close proximity to each other, is located at the proximal end. From its widest point, in the larger proximal area 14, the profile continues toward a distal end 16 in a narrowing fashion wherein gripping features 18 are provided around the circumferential profile. Moving toward the distal end, the profile tapers outward from a transition point 19 adjacent to gripping features 18. The gripping features 18, in this embodiment are shown as a series of concavities spaced apart from each other around the periphery of a handle 10 in the region between the larger proximal area 14 and the transition 19. It should be understood by those recently skilled in the art that although these gripping features are shown here as a series of concavities, they may take other shapes such as depressions, or projections of various shapes and may alternately be formed of different materials which facilitate hand gripping by the surgeon. At the distal end 16, a series of secondary bearing surfaces 17 are located around the circumference thereof. The secondary bearing surfaces 17 shown in this embodiment are shaped as semi-spherical concavities to receive balls 52 and it should be understood by those reasonably skilled in the art that the shape of the secondary bearing surfaces 17 should be selected to accommodate the component against which it bears, in this case a ball 52. Other shapes for the secondary bearing surfaces 17 are therefore contemplated and within the scope of the invention. In this embodiment, the handle 10 is molded from a plastic material, however, it should be understood by those reasonably skilled in the art that other suitable materials, molding and/or machining techniques may be employed to fabricate a handle 10.

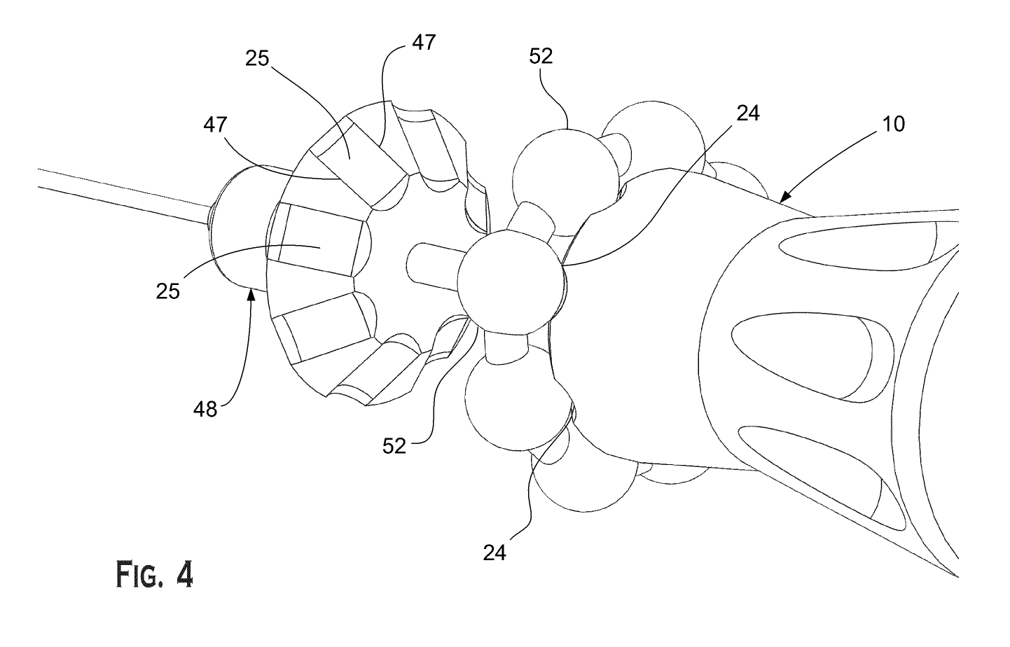

Next, the spring housing assembly 20 will be described in greater detail. The spring housing assembly 20 consists of a spring housing 22, a spring 36, a spacer 34, a distal bushing assembly 32, and a proximal bushing assembly 38. The spring housing 22 is a generally cylindrical component having a series of primary bearing surfaces 24 arranged along an integral flange 26 positioned along and angled toward the distal end 28. The primary bearing surfaces 24 are designed to have concavities 25 around the circumference thereof. As best shown in FIG. 4, in this embodiment, the concavities 25 are generally U-shaped channels and are designed to receive the balls 52 along edges 24 thereof. Such edges 24 act as rails upon which the balls 52 ride. Therefore, the concavities 25 may take other shapes which are suitable for receiving and easily gliding the balls 52 as will be described below. It should be understood by those reasonably skilled in the art that the shape of the primary bearing surfaces 24 should be selected to accommodate the component against which it bears, in this case a ball 52. Other shapes for the primary bearing surfaces 24 are therefore contemplated and within the scope of the invention. A first opening 21 and the second opening 23 are formed in the wall of the spring housing 22 near its proximal end. In this embodiment, the spring housing 22 may be formed by molding or machining any suitable plastic or metallic material. The spring 36 is formed as a coil spring and is positioned inside the spring housing 22 against the integral flange 26 on its distal end 36d and, on its proximal end 36p, against a spacer 34 which takes the form of a flat washer. The distal bushing assembly 32 is generally cylindrical and has a set screw 31 passing through a bushing wall thereof and secured thereto with threads. The distal bushing assembly 32 is hollow such that it has a tube receiving passageway 33 extending from its distal end to its proximal end. The set screw 31 may be advanced from the bushing wall into the tube receiving passageway 33. The proximal bushing assembly 38 likewise consists of a bushing 39 which is generally cylindrical and has a pair of set screws 37 which are threaded into and pass through the bushing wall. The bushing 39 is hollow such that it has a tube receiving passageway 35 extending from its distal end to its proximal end. The set screws 37 may be advanced from the bushing wall into the tube receiving passageway 35.

Next, the ball ring assembly 50 will be described in greater detail. The ball ring assembly 50 consists of a plurality of balls 52 and a ring 54. Each of the balls 52, is a generally spherical component having a generally cylindrical passageway 56 formed therein. The passageway 56 may take a cylindrical shape or may alternatively be formed to have a slight arcuate shape. In this embodiment, the balls 52 are made of a plastic material, however, they may be formed of other suitable materials such as metal, glass, Teflon or any other material which glides freely between the primary bearing surfaces 24, 47. The ring 54 is made of a flexible material such as silicone tubing but may be made of any other suitable flexible tubular material. In this embodiment, the ring 54 has a circular cross section but it should be understood by those recently skilled in the art that the cross-section may be modified and adapted to various different passageway 56 shapes. For example, the ring 54 may have a square or rectangular cross-section. The ball ring assembly 50 is assembled by threading the ring 54 through each of the passageways 56 and then joining by bonding, fusing or any other suitable method the respective ends of the ring 54 together. Once assembled, the ball ring assembly 50 remains somewhat flexible and/or stretchable.

Turning now to the distal assembly 40, it consists of an inner tube 42 , an outer tube 44, a cap bushing 46 and a cap 48. The cap 48 has a generally conical shape and has an opening passing through its center from a distal end to a proximal end thereof. Along the proximal end, a plurality of bearing surfaces 47 are located and are similarly shaped to primary bearing surfaces 24 such that they form a pair of rails for each ball 52 to ride upon. The bearing surfaces 47 face toward the spring housing assembly 20. The cap 48, in this embodiment, is formed of the same material as the spring housing 22 but it should be understood by those reasonably skilled in the art that it may be formed of any suitable molded or machined material. The inner tube 42, in this embodiment, formed of a surgical stainless steel material, has a smaller diameter than the outer tube 44, in this embodiment, also formed of the same material. The inner tube 42 is designed to fit into the outer tube 44. The cap bushing 46 is profiled to fit within an end of the outer tube 44 with an interference fit. The inner tube 42 fits tightly within the cap bushing 46 to secure both the inner and outer tube together. In this embodiment, the cap bushing 46 is made of a stainless steel material, however, it should be understood that the cap bushing may be made of other suitable materials that form an interference fit between the inner and outer tubes 42, 44.

The operative tool 60 consists of a rod 62 having a tool 64 integrally formed on its distal end. In this embodiment, the tool 64 is a set opposing jaws designed to open and close upon actuation as will be more fully described below. The rod 62 has a diameter and is profiled to slidingly fit within the inner tube 42. The operative tool 60 is made of a suitable surgical metal, however it should be understood by those reasonably skilled in the art that other materials suitable for a selected tool 64 may be substituted.

Finally, the optional handle extension 70 is a generally cylindrical component having a threaded portion 72 at its distal end and a rounded end 76 at its proximal end. It may alternatively have a proximal opening with a groove that accommodates a raised ring in the distal end to form a snap fit between handle 10 and optional extension handle 70. A gripping section 74 is located between the threaded portion 72 and rounded end 76.

Now, assembly of the instrument 1 will be described in greater detail. The distal assembly 40 is assembled by inserting the inner tube 42 into the cap bushing 46 and then inserting the two of them into the outer tube 44 whereby the three components are frictionally fit together. Alternatively, they may be otherwise bonded or soldered together.

The spring housing assembly 20 is assembled by inserting the spring 36 into the proximal end of the spring housing 22 followed by insertion of the spacer 34 behind the spring and the distal bushing without the set screw 31 inserted behind the spacer 34 such that the distal bushing assembly 32 has its threaded opening aligned with the first opening 21 in the spring housing assembly 20. Next, the preassembled distal assembly 40 is placed against the spring housing 22 with the ball ring assembly 50 positioned therebetween such that balls 52 are located within bearing surfaces 24, 47. The inner and outer tubes 42, 44, moving in unison, are urged into the spring housing 22 until the proximal end of the outer tube 44 passes through the distal bushing assembly 32. The set screw 31 is inserted through the first opening 21 and threaded through the distal bushing assembly 32 to secure the outer tube 44 therein.

Next, the rod 62 of the operative tool 60 is inserted through the tubes 42, 44 until it passes through the spring housing 22 and exits the proximal end thereof. The proximal bushing assembly 38 is then secured to the proximal end of the rod 62 by application of the two set screws 37. The proximal bushing assembly 38 is then pressed into and/or bonded to, or otherwise secured to, the proximal end of the spring housing 22. Handle 10 is then applied over the proximal end of the spring housing 22 and secured by a frictional fit or suitable bonding technique such as adhesive, welding, ultrasonic bonding or any other suitable method. The optional handle extension 70 may be threaded into the threaded opening 13 of the handle 10 or alternatively snap fit as described above. The optional handle extension 70, in this embodiment, may also be used as an instrument guard by sliding it's open end over the operative tool 60 such that the open end slides over and engages the distal end of the cap 48.

Now, operation of the instrument will be described in greater detail. Referring to FIG. 2, the instrument 1 is shown in its unactuated position wherein the tool 64 protrudes from the inner tube 42. The instrument 1 is actuated by axially moving the balls 52 forward by pinching or squeezing the rear of the ball ring assembly 50. This action applies an axial force to the bearing surfaces 47, 24 and 17 urging cap 48 and spring housing assembly 20 apart from each other. The inner tube 42 and outer tube 44 move in unison with the cap 48 toward the tool 64 whereby the inner tube 42 slides over the rod 62. Motion of the outer and inner tubes 44, 42 is limited by the proximal bushing assembly 38. As shown in FIG. 2, the set screw 31 protruding from the distal bushing assembly 32 travels within the first opening 21 of the spring housing assembly 20. The outer tube 44, being secured to the distal bushing assembly 32 has a limited range of motion defined by the size of the first opening 21. It should be understood that in the unactuated position shown in FIG. 2, the spring 36 biases the set screw 31 against the proximal end of the first opening 21 and force applied by depression of the ball ring assembly 50 urges the set screw toward the distal end of the first opening 21 against the spring bias force. Upon actuation, the inner tube 42 engages the tool 64 in order to close its jaws as the tube moves toward the distal end. Releasing depression of the ball ring assembly 50 causes the distal assembly 40 to move back towards the proximal end under the spring force.

It should be understood that while the invention has been described here in view of an exemplary embodiment, other embodiments having variations on the concepts are contemplated and intended to be within the scope of the invention.

What is claimed is:

1. A surgical instrument comprising:
a handle;
a spring housing assembly disposed at a distal end of and secured within the handle;
a distal assembly disposed against the spring housing assembly;
a ball ring assembly having a plurality of balls and positioned between the spring housing assembly and the distal assembly; and
an operative tool inserted through the distal assembly and connected to the spring housing assembly.

2. The surgical instrument according to claim 1, wherein the handle includes a plurality of secondary bearing surfaces positioned around a circumference of a distal end.

3. The surgical instrument according to claim 2, wherein the plurality of secondary bearing surfaces are semi-spherical concavities to receive the plurality of balls.

4. The surgical instrument according to claim 1, wherein the spring housing assembly includes a spring housing and a spring, the spring inserted through a proximal end of the spring housing.

5. The surgical instrument according to claim 4, wherein the spring housing is a cylindrical component having a plurality of primary bearing surfaces arranged along an integral flange positioned along and angled toward the distal end.

6. The surgical instrument according to claim 5, wherein the plurality of primary bearing surfaces are concavities for receiving and engaging with the plurality of balls.

7. The surgical instrument according to claim 5, further comprising a first opening and a second opening formed in a wall of the spring housing.

8. The surgical instrument according to claim 4, wherein the spring is a coil spring positioned inside and abutting an integral flange of the spring housing.

9. The surgical instrument according to claim 1, further comprising a distal bushing assembly having a set screw passing through a bushing wall.

10. The surgical instrument according to claim 9, wherein the distal bushing assembly includes a first tube receiving passageway extending from a distal end to a proximal end.

11. The surgical instrument according to claim 10, wherein the distal assembly extends into the first tube receiving passageway and secured by the set screw.

12. The surgical instrument according to claim 10, further comprising a proximal bushing assembly positioned in the spring housing assembly and having a set screw.

13. The surgical instrument according to claim 12, wherein the proximal bushing assembly includes a second tube receiving passageway extending from the distal end to the proximal end.

14. The surgical instrument according to claim 13, wherein the operative tool extends through the first and second tube receiving passageways and is secured by the set screw.

15. The surgical instrument according to claim 1, wherein the ball ring assembly includes a flexible ring extending through the plurality of balls.

16. The surgical instrument according to claim 15, wherein each ball of the plurality of balls is a spherical component having a cylindrical passageway formed therein for receiving the flexible ring.

17. The surgical instrument according to claim 16, wherein the ball ring assembly is assembled by threading the flexible ring through the cylindrical passageway of each ball and then joining respective ends of the flexible ring together.

18. The surgical instrument according to claim 1, wherein the distal assembly includes an inner tube, an outer tube, and a cap, the inner tube fits into the outer tube which extends into the cap.

19. The surgical instrument according to claim 18, wherein the cap is conical and having an opening passing for receiving the outer tube.

20. The surgical instrument according to claim 19, wherein the cap includes a plurality of distal bearing surfaces positioned across and similarly shaped to a plurality of primary bearing surfaces arranged along an integral flange disposed along the spring housing assembly and angled toward the distal end.

21. The surgical instrument according to claim 20, wherein the plurality of distal bearing surfaces and the plurality of primary bearing surfaces provide a plurality of rails for the plurality of balls to be positioned and engage there with.

22. The surgical instrument according to claim 21, further comprising a cap bushing that interference fits within an end of the outer tube.

23. The surgical instrument according to claim 22, wherein the inner tube fits tightly within the cap bushing to secure both the inner and outer tube together.

24. The surgical instrument according to claim 1, wherein the operative tool includes a rod having a tool integrally formed on a distal end of the operative tool.

25. The surgical instrument according to claim 24, wherein the tool is a set opposing jaws designed to open and close upon actuation.

26. The surgical instrument according to claim 25, wherein the rod slides within an inner tube of the distal assembly.

27. The surgical instrument according to claim 1, further comprising an optional handle extension.

28. The surgical instrument according to claim 27, wherein the handle includes a threaded opening that receives a distal end of the optional handle extension to form a snap fit between the handle and the optional handle extension.
